(19) United States
(12) Patent Application Publication
(10) Pub. No.: US 2016/0287814 A1
(43) Pub. Date: Oct. 6, 2016

(54) MEDICAL ILLUMINATION DEVICE

(71) Applicant: JENNUS INNOVATION CORPORATION, Mendota Heights, MN (US)

(72) Inventors: Timothy G. McManus, Mendota Heights, MN (US); Alexander R. Jenny, Wayland, MA (US); Michael E. O'Neill, Randolph, MA (US)

(21) Appl. No.: 15/091,709

(22) Filed: Apr. 6, 2016

Related U.S. Application Data
(60) Provisional application No. 62/143,358, filed on Apr. 6, 2015.

(51) Int. Cl. A61M 5/42; A61B 5/00

(52) U.S. Cl. CPC A61M 5/427; A61B 5/0084; A61M 2205/8206

(57) ABSTRACT

A device for locating blood vessels of a patient includes at least one illumination strip including a top surface and a bottom surface. There is a securing device connected to the at least one illumination strip and configured to affix the bottom surface to the patient's skin. There is a light source disposed along the at least one illumination strip and directed toward an area on the patient's skin adjacent to the at least one illumination strip to illuminate the area on the patient's skin and enhance visibility of the patient's blood vessels.

### CROSS REFERENCE TO RELATED APPLICATIONS

This application claims priority to U.S. Provisional Application No. 62/143,358, filed on Apr. 6, 2015, the contents of which are hereby incorporated by reference in its entirety.

### FIELD OF THE INVENTION

This invention relates generally to medical imaging, and more particularly to a medical device, which illuminates vasculature and soft tissue in the human body. The device is especially useful for efficiently identifying suitable subcutaneous veins for venipuncture and other medical procedures.

### BACKGROUND OF THE INVENTION

Venipuncture is a procedure performed in medicine by which a hollow needle is passed through a person's skin and into the vasculature of the body to achieve direct access to the bloodstream. The procedure is frequently utilized in a number of healthcare settings for a variety of reasons, but most venipunctures are performed for obtaining blood samples for diagnostic lab values and placing intravenous (IV) lines that deliver rapid, systemic therapies. IV lines are also frequently utilized for correcting electrolyte imbalances, infusing appropriate fluids for rehydration, performing blood transfusions, and administering pharmaceutical therapies such as chemotherapy regiments.

Due to the intrinsic role that venipuncture plays in patient care, it is critically important that the procedure is carried out as efficiently and safely as possible. Nurses, phlebotomists, and other healthcare workers traditionally identify suitable veins by touch and unaided eye. Many patient types with less superficially visible subcutaneous veins can pose difficulties for the administrators of intravenous lines, patients, and the hospital or lab. The literature suggests that there are increased risks and complications for venipuncture misses (i.e., missing the intended vein), which are more frequent in certain patient populations including, but not limited to: pediatrics, patients who experience chronic venipuncture (e.g., chemotherapy patients), obese, advanced age, dark skin pigmentation, edematous, and trauma. Other causes for difficulty locating suitable veins for venipuncture include: dehydration, hypotension, peripheral vasoconstriction, poor vein quality, telangiectasia, skin rash, IV drug use, and low skill of technician.

When venipuncture misses occur, the perforating needle can cause unwanted complications such as nerve damage, artery puncture, and other soft tissue injury. Multiple venipuncture attempts to place an IV line also increases the risk of infection. IV infection is a serious complication since patients can develop into septicemia, which dramatically worsens patient outcomes and increases morbidity rates. The cost to treat IV infections is also extremely high as hospital acquired infections are often not fully reimbursed by insurance. This leaves hospitals or clinics absorbing the cost of treating the infection. The costs of venipuncture misses also escalate when specialists or physicians need to be called in to perform the procedure. The increased amount of time it takes to find specialists and physicians holds an associated cost and introduces additional inefficiencies into patients' healthcare.

In addition to the adverse effects that venipuncture misses have on patients' quality and cost of care, they also have a profound impact on their overall perception of the care that they receive. The quality of the patient experience will play an increasingly important role in healthcare delivery as the Center for Medicare & Medicaid Services has established The Hospital Consumer Assessments of Healthcare Providers and Systems, which collects information on patients' perspectives of care and establishes a standard metric for measuring patient satisfaction. Under the Affordable Healthcare Act, these patient satisfaction scores will be used to help allocate reimbursement dollars.

One of the most impressionistic aspects for patients in charting their patient satisfaction is their venipuncture experience. Venipuncture is a critical component to the patient experience as it is one of the first procedures performed on patients visiting hospitals. It is also an experience that patients easily recall since they are usually awake when they are subjected to this often painful and stressful procedure without any numbing or prophylactic measures to lessen the pain or discomfort. Additionally, a number of patients suffer from a condition called trypanophobia, or “needle phobia,” meaning an individual has an intense fear of needles, which can result in physical responses including, but not limited to: increased heart rate, rise in blood pressure, stress hormonal release, syncope, and acute psychosomatic episode.

Illuminating patients' veins to assist in the delivery of an IV can reduce venipuncture misses, increase patient satisfaction, and reduce costs for a hospital or lab. The practice of illuminating patients' veins is known in the field. A number of technologies and manifestations of this practice are common today, but they are either prohibitively expensive for hospitals and labs, or far less effective than the present invention.

For example, solutions such as Veinlite LED (U.S. Pat No. 20120101343 A1), shown in FIG. 1, use LEDs in a handheld device to illuminate veins on patients' skin. Similar products include AccuVein, FIG. 2, and Vein Viewer from Christie Medical Holdings. The Vein Viewer is offered in both a handheld solution as well as through a bedside attachment. Other solutions exist for vein illumination as well, including Vein-Eye, from Near Infrared Imaging, which provides a similar solution, though the veins are projected onto an electronic monitor, rather than illuminated on patients' skin. Ultrasound technology is also used to access veins. Bard Access Systems provides a solution called Prevue, which creates images of veins on a small monitor via ultrasound. While these solutions have proven to be effective for vein illumination, all of them are prohibitively expensive, particularly to hospitals with lower IV volumes.

Existing less costly solutions are also less effective. One existing disposable patch is described in U.S. Pat. No. 7,925,332, which is depicted in FIG. 3. This product is used on the side of the arm or hand that is opposite the IV insertion site. Another lesser expensive alternative is an LED pen, such as the Streamlight Stylus Pro, but this solution is far less effective than the present invention, which incorporates a disposable patch with additional features that improve venipuncture and the patient experience.

Once an IV is inserted, it is critically important to properly secure and stabilize it. Currently, IVs are secured with a wide range of products ranging from tape to aseptic dressings, such as 3M's Tegaderm™ and IV securement kits from Centurion Medical Products. These products come in many shapes and sizes, and may or may not include an aseptic material to help reduce the risk of infection. Studies have shown that catheter securement devices are far more effective at securing and stabilizing an IV line than tape, though tape is still very common. Using excess tape can block visibility of the IV site, which may block any visual signs of infection or other complication. One study estimated that between 40 and 70 percent of all Peripheral Intravenous Catheter (PIVC) insertions are caused by unscheduled restarts (i.e., replacing a failed PIVC). IV restarts require additional time for healthcare workers, and again increase risks and complications. If an IV is not secure, the patient may face an increased exposure to infection.

### SUMMARY OF THE INVENTION

In one aspect, the invention features a device for locating blood vessels of a patient including at least one illumination strip having a top surface and a bottom surface. There is a securing device connected to the at least one illumination strip and configured to affix the bottom surface to the patient's skin. There is a light source disposed along the at least one illumination strip and directed toward an area on the patient's skin adjacent to the at least one illumination strip to illuminate the area on the patient's skin and enhance visibility of the patient's blood vessels.

In other aspects of the invention one or more of the following features may be included. There may be a plurality of interconnected illumination strips. The plurality of interconnected illumination strips may form a frame which defines a central aperture about the area on the patient's skin and the plurality of interconnected illumination strips may each include a light source directed toward the central aperture. The securing device may comprise an adhesive disposed on the bottom surface of at least one illumination strip to affix the device to the patient's skin. The securing device may comprise an adhesive disposed on the bottom surfaces of the illumination strips to affix the device to the patient's skin. The securing device may comprise a strap configured to be disposed about a limb of the patient and secured by a fastener to affix the device to the patient's skin. There may be included a membrane hinged to a first portion of the frame and configured to be folded between an open position and a closed position to cover the central aperture and secure an intravenous line in place after insertion of the intravenous line into the patient's blood vessel through the central aperture. There may be a power source configured to provide power to said light source. There may be a switch interconnected between the power source and the light source to activate the light source and illuminate the area on the patient's skin. The power source may comprise a battery activated by a pull tab. The battery may be affixed to a portion of at least one illumination strip and the battery may include a top surface on which a label is disposed to record patient information.

In further aspects of the invention one or more of the following features may be included. The light source may comprise a plurality of light emitting diodes and the light emitting diodes may comprise printed strips of near infrared and other wavelengths of light emitting diodes. The light source may comprise a plurality of light emitting diodes disposed about the border of the frame. The at least one illumination strip may comprise a fluorescent filter to enhance illumination. The bottom surfaces of the plurality of illumination strips may contain an aseptic agent. The aseptic agent may include one or more of chlorhexidine gluconate, silver and zinc particles, and iodine. The membrane may contain an aseptic agent and the aseptic agent may include one or more of chlorhexidine gluconate, silver and zinc particles, and iodine. The frame may be rectangular in shape. The at least one illumination strip may be in the form of a band which wraps around a limb of the patient. The light source may be disposed above the top surface of the illumination strip. There may further be included at least one additional illumination strip which extends across the central aperture. The at least one illumination strip which extends across the central aperture may be curved in shape. There may further be included a sterile package which encompasses the device. The device of claim 1 further including a bumper disposed on the bottom surface of the at least one illumination strip to acts as a tourniquet to pool blood in the blood vessels.

In other aspects of the invention one or more of the following features may be included. There may be further included a fluorescent filter to enhance illumination of the light source. There may be included an ambient light filter. The light source may emit light a first wavelength suited to penetrate the skin and a second wavelength which allows the blood vessels to absorb the light. There may further be included a filter comprising materials to enhance viewing of the the second wavelength.

### BRIEF DESCRIPTION OF THE DRAWINGS

Referring to the drawing, wherein like numerals represent like parts throughout the several views:

FIG. 1 is a perspective view of a prior art handheld vein illumination device;

FIG. 2 is a perspective view of a prior art vein illumination device that attaches to chair or patient bed;

FIG. 3 is a view of the veins in a patient's arm illuminated with a prior art disposable patch placed on underside of the arm;

FIG. 4 is a perspective view of an embodiment of the medical illumination device according to an aspect of this invention;

FIG. 5 is a series of perspective views depicting the use of the medical illumination of FIG. 5;

FIG. 6 is top down view of another embodiment of the medical illumination device according to an aspect of this invention;

FIG. 7 is a perspective view of another embodiment of the medical illumination device according to an aspect of this invention;

FIG. 8 is a perspective view of yet another embodiment of the medical illumination device according to an aspect of this invention;

FIG. 9 is top down view of another embodiment of the medical illumination device according to an aspect of this invention;

FIG. 10 is a perspective view of yet another embodiment of the medical illumination device according to an aspect of this invention;

FIG. 11 is top down view of yet another embodiment of the medical illumination device according to an aspect of this invention with the capability to illuminate in sterile, packaged environment;

FIG. 12 is a side elevational view of another embodiment of the medical illumination device of an aspect of this invention with a raised the lighting source;

FIG. 13 is a top down view of the bottom surface of the medical illumination device according to an aspect of this invention depicting the adhesive portions of the device;

FIG. 14 is a side elevational view of another embodiment of the medical illumination device of an aspect of this invention with tourniquet bumper; and

FIG. 15 is an underside view of the medical illumination device of FIG. 14.

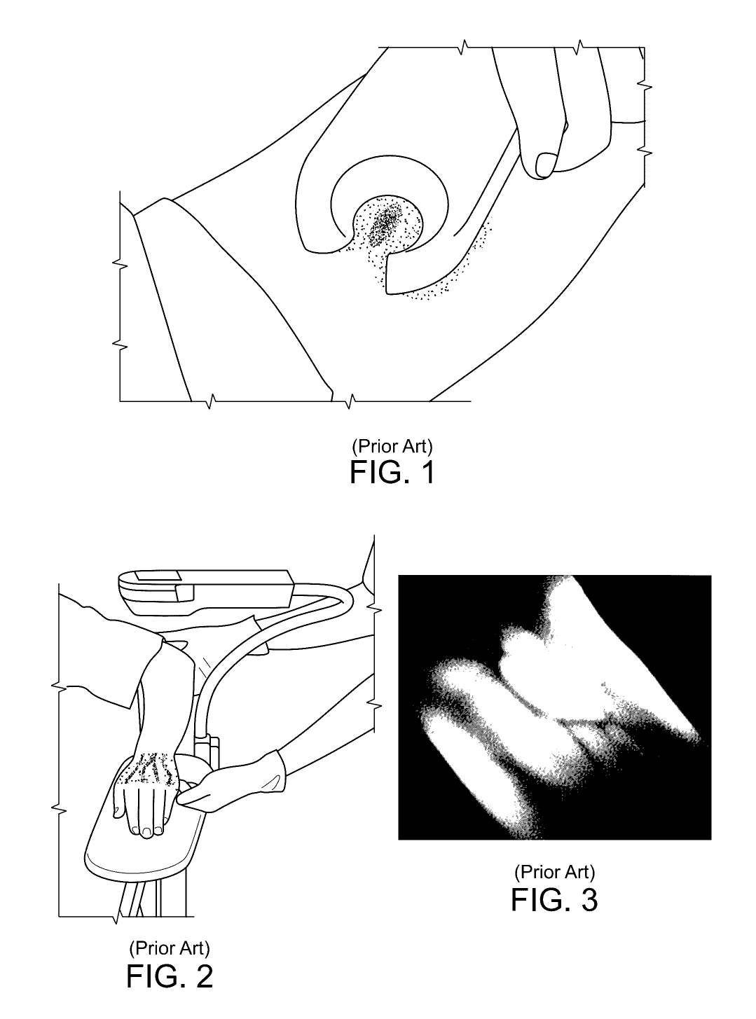

### DETAILED DESCRIPTION

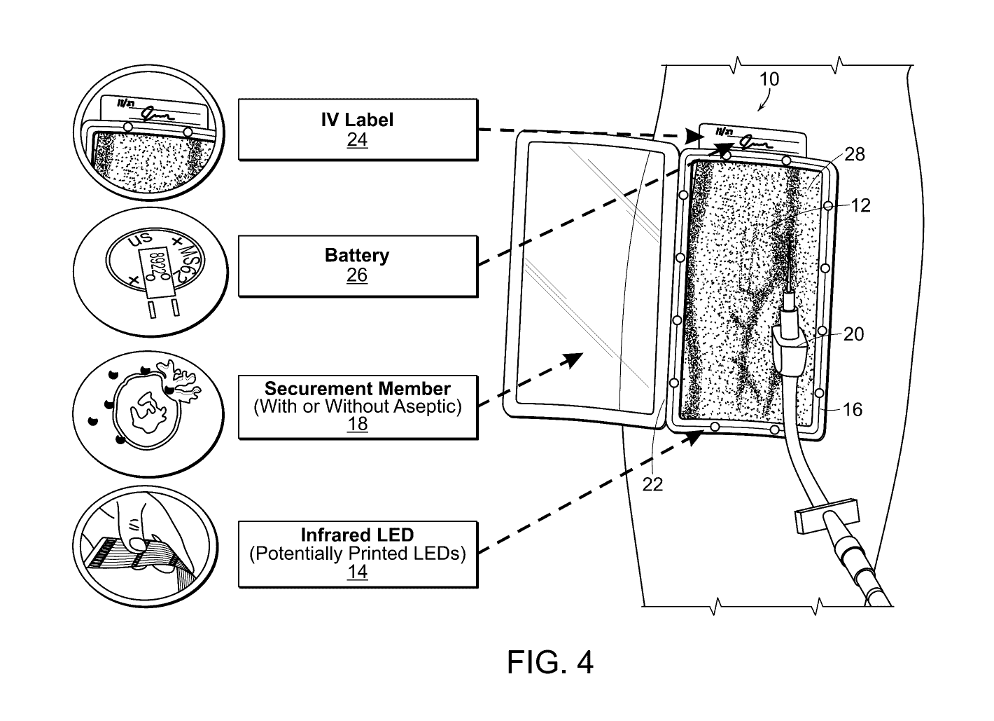

The present invention provides an improved configuration applicable to many clinical settings. The invention provides a medical illumination device 10 as shown in FIG. 4 that enhances the visualization of subcutaneous vasculature 12 and soft tissue in the body. Venipuncture procedures in the past have primarily relied on healthcare workers' ability to identify a vein by touch and unaided eye. The introduction of vein illumination devices, as previously mentioned, have made these healthcare workers' job of locating veins easier, but these devices have seen some resistance in the market place.

One notable deficiency of such devices is that they are often cumbersome to use. As many are in a handheld format, they may require the assistance of an extra healthcare worker to hold to the device while a different worker inserts the IV. Other solutions can be clipped onto patient's beds or are a part of a larger platform on wheels. Some solutions require the use of a computer monitor to project the images of the subcutaneous veins taken with a camera. These solutions are often very expensive, and frequently this cost can prohibit the purchase for a hospital or clinical lab. Finally, these solutions require ongoing maintenance and service if there are any technical issues. If the device were to malfunction, it would be unusable until the supplying company could service it. Additionally, many of these devices must be charged, require the continued refreshing of batteries, or require access to an AC power source during use, which can limit the locations and times where it may be utilized.

The invention presents an alternative to these existing vein illumination solutions through a versatile, cheaper, easier-to-use, and hands-free patch that is placed on the patient's arm via an adhesive, while providing additional capabilities. The invention allows the medical worker to insert the IV while the patch is adhered to the patient. The worker may insert the IV either within a ring of light, or adjacent to the light source, into an illuminated vein. Once the patch is placed on the arm and the LEDs are switched on (powered by a small, low-powered battery), the IV insertion process begins. The worker inserts the IV inside the LED illumination frame patch perimeter into a now-illuminated vein. Upon completion of the IV insertion, the LEDs are switched off. One version of the embodiment is a dual patch system which has an illumination component, but also a securement dressing which can be folded over the IV (and illumination frame patch) to secure the IV. The invention may be kept on the patient so long as the IV must be present. Another embodiment of the invention allows for an antiseptic solution on the securement patch to keep the area clean. When the IV is ready to be removed, the invention can be removed and disposed of. This is an ideal attribute of the enclosed invention as it allows for easy use and concurrently reduces the risk for spreading microbes and other unwanted microorganisms which can lead to infection.

The invention provides a simple yet effective mechanism for illuminating subcutaneous veins, or at least significantly enhancing the visualization of such veins, leading to improved venipuncture access, patient satisfaction, and decreased risks and complications. Improving venipuncture stick success rates will lower the costs associated with venipuncture escalations. Using the enclosed medical illumination invention cuts cost and will also produce higher patient satisfactions scores for hospitals. The present invention is more versatile than current solutions. Given the lower cost and smaller size of the invention, as compared to current illumination solutions, it can be placed in many areas of a hospital or lab that may require assistance during IV insertion. Current solutions can only be used in one place, at one time, and must be carried or wheeled to a patient (or the patient must travel to that location if the facility uses an immobile solution). Current solutions must also be sterilized before and after use which presents maintenance and upkeep requirements. It also increases the risk of spreading infection. The enclosed invention is useful in ambulatory or other field medicine settings.

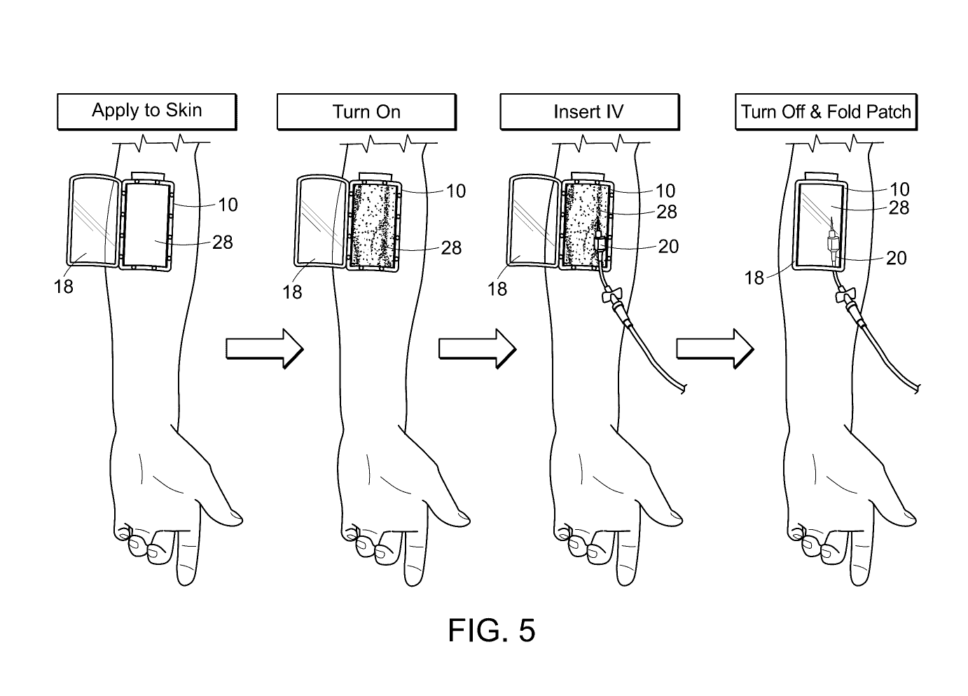

Referring to FIGS. 4 and 5, the invention is a trans-illumination device that may be laid atop or applied to the skin. The preferred embodiment allows for a single-use, dual system patch that is equipped with both vein illumination technology in the form of light emitting diodes 14 disposed about an illumination frame 16 which is disposed on the patient's skin. Illumination frame 16 may be in the form of a thin membrane made of a synthetic material such as a polymer or plastic. Other materials might include fiberboard, paper or rubber. There may be a securement member 18, which, along with illumination frame 16, may be comprised of a thin, breathable membrane with an adhesive surface that is flexible for greater comfort when applied to the body. The adhesive properties of the illumination frame 16 and securement member 18 allow for the device to be adhered to the skin of a patient. There may be various configurations of the adhesive on the bottom surface of the illumination frame 16 and securement member 18. A gel pad containing aseptic agents (e.g., chlorhexidine gluconate, silver and zinc nanoparticles, iodine) may be integrated into both the securement member as well as the illumination frame, which serves as the invention's aseptic environment enforcement. The adhesive component of the illumination frame serves as the anchoring portion of the invention. The illumination frame is typically applied to the skin first in order to illuminate vasculature (i.e. blood vessels) via LEDs 14. Once the veins have been visualized and the IV 20 properly inserted, the securement member 18 is then folded over to hold the IV 20 in place. The securement member 18 is affixed to the illumination frame 16 in a hinge type fashion as shown at hinge 22 which allows for the adhesive securement member to simply fold over the inserted IV 20 (and subsequently, the illumination frame 16). The securement member not only aids in contamination prevention, but it also stabilizes and secures the IV. Finally, the invention may also incorporate an IV label 24, to add an additional element of functionality and usefulness. The IV label 24 may be adhered to the bottom, middle, or on the top surface of the illumination frame membrane or securement member. The IV label is where medical workers scribe vital patient information while the IV is in use.

The invention's illumination technology may be comprised of printed or non-printed LED lights 14 in the form of strips, which may be layered into the membrane (formed of a translucent plastic or other suitable material) of the illumination frame 16. In this embodiment, the illumination frame 16 is in the form of a rectangle but other shapes, some of which are described below, may be used. The light source may emit a range of light of selected wavelengths (e.g. infrared and/or visible light) which are absorbed by the vasculature (specifically the hemoglobin contained within a blood cell) whereas the surrounding body tissues does not absorb the light. This effectively allows for visualization of the vasculature as it becomes illuminated allowing for the vasculature to be differentiated from the surrounding body tissues. The light source may be layered into the membrane of the illumination frame in a configuration by which the light source is attached to the membrane on the bottom surface or top surface. Another embodiment for the light source attachment to the membrane includes a configuration where the light source is embedded into the membrane with portions of the light source diode visible on the bottom and upper surfaces of the membrane. The light source (LED or other light sources) may be constructed into the membrane of the illumination patch in a fashion where the light emission (i.e. light diode or light bulb) has a directionality that is angled or aimed downward towards the skin. Furthermore, the light emission from the light source can also be angled or aimed in a myriad of directional planes including, but not limited to: oblique, lateral, medial, superior, and inferior directionality and/or any combination thereof that may enhance the visualization of vasculature. The membrane illumination frame 16 may be coated with a fluorescent filter to maximize vein illumination potential. The LED lights are powered by a small battery 26 which may be operated by a simple pull-tab switch. In this embodiment label 24 covers the battery 26 and allows healthcare workers to efficiently chart initials, IV insertion date, and any other documentation.

FIGS. 4 and 5 present the various components of the preferred embodiment of the invention. The portion outside of the red, illuminated segment of skin is adhered to the patient, while the other “flap” is used to secure the IV once inserted to the patient. The LEDs or other lights are secured onto a second layer attached to this ring of adhesive. This second layer allows for separation from the patient's skin. FIG. 4 shows use of the medical illumination device 10 after IV 20 has been inserted, while the lights are illuminated, and before the IV is secured with the securement patch. FIG. 5 shows the steps in the process of using medical illumination device 10.

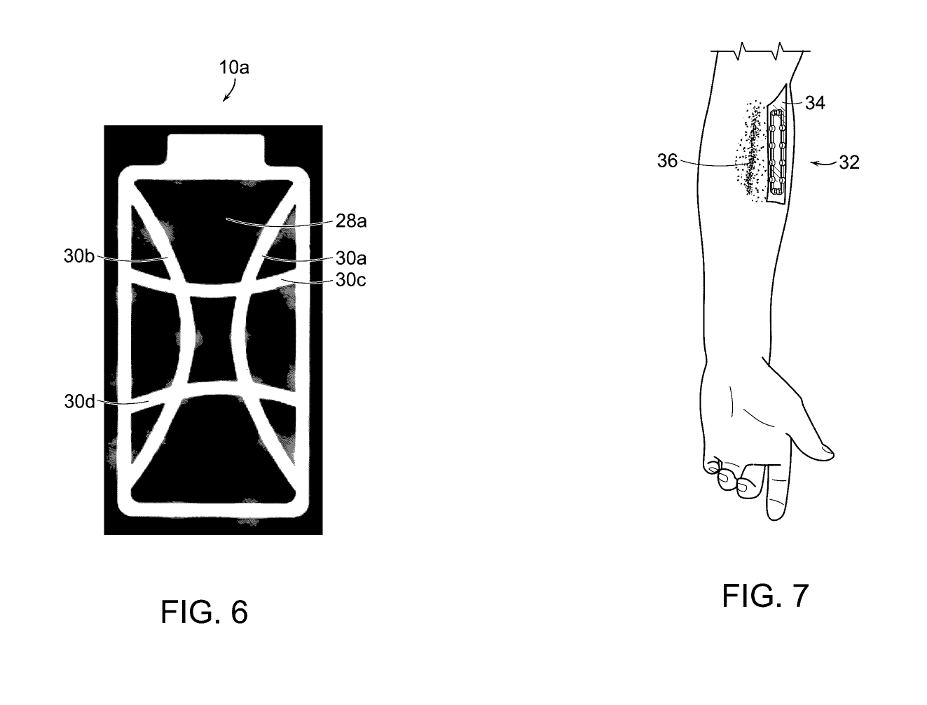

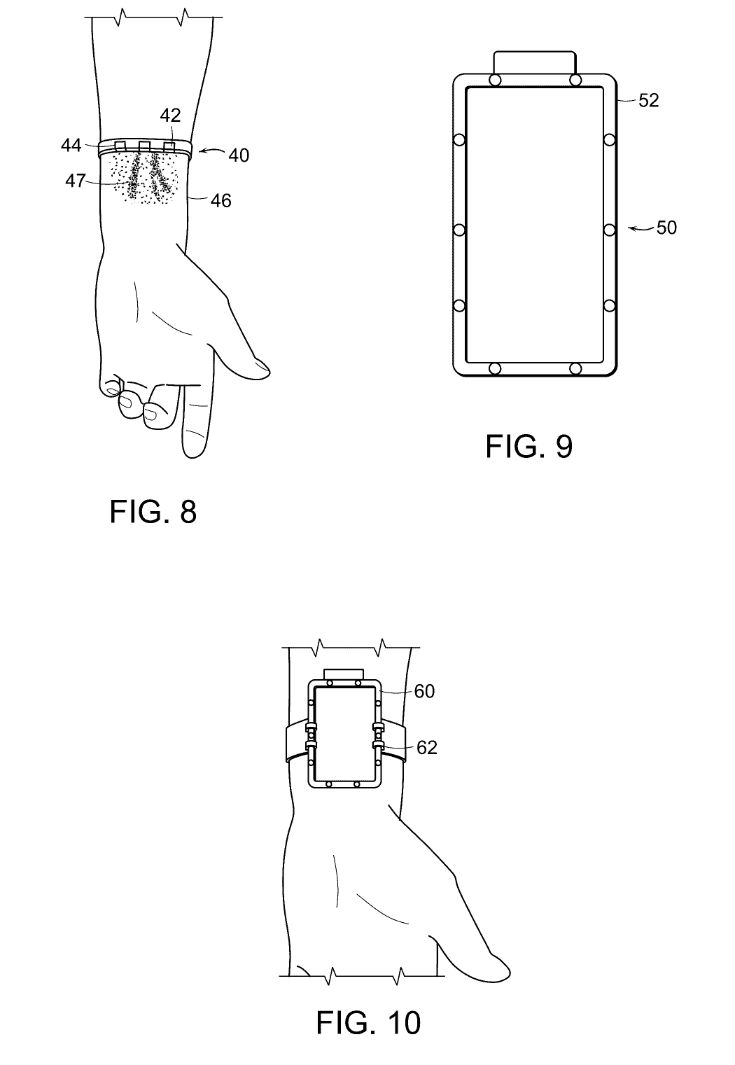

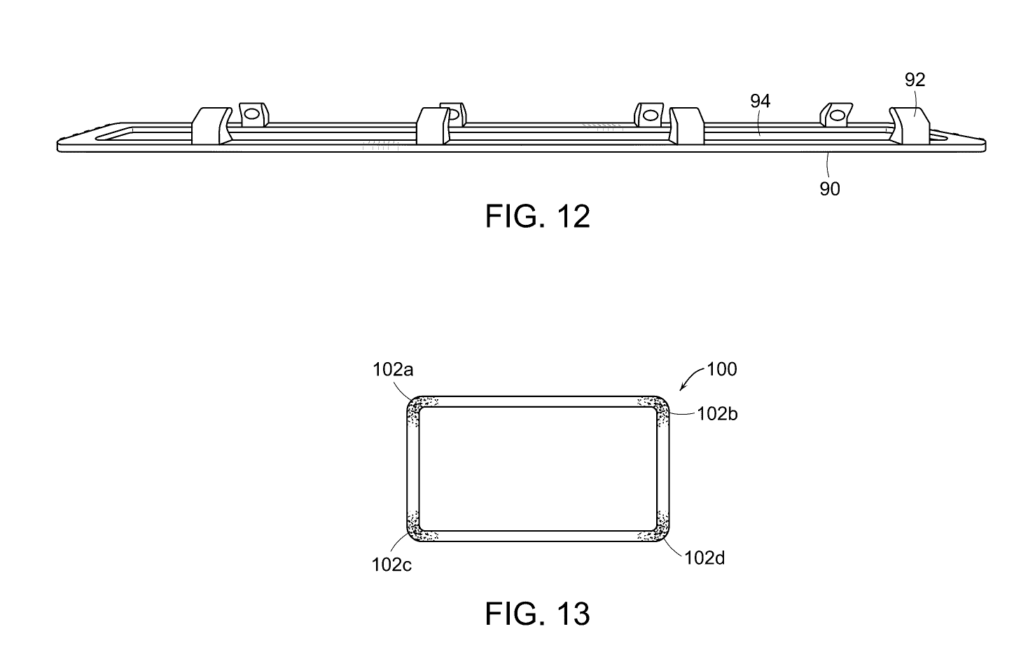

Additional embodiments may include, but are not limited to hose depicted in FIGS. 6-15 below:
Various layouts of LEDs (e.g., concentric ring, rectangle, diamond shape, line segment) (FIG. 6,7,8);
Securement dressing with or without aseptic agents or material;
Illumination patch with no IV securement functionality, for use in one-time blood draws or injections (FIG. 9);
Various methods of placing the invention on patients' bodies, other than with an adhesive solution (e.g., an armband or wristband) (FIGS. 8 &10);
Reusable portion of the invention (e.g., reusable power source or LED lights), or an entirely reusable version of the invention;
Various configurations, shapes, and sizes of the securement dressing;
Various configurations, shapes, and sizes of the entire patch (e.g. armband or wristband configuration (FIG. 9, 10);
Various configurations of LEDs, printed LEDs, or other light sources to illuminate veins in preparation of IV insertion with various wavelengths and colors; including a raised and/or elevated configuration (FIG. 12)
Various configurations of the adhesive component of the illumination patch, may be continuous with the patch, segmented, etc. (FIG. 13)
Additional power sources (e.g., AC power extension, chemical reactions, and non-battery operated power sources);
Additional mechanisms in addition to pull tab for initiating power source (i.e., button turn on and off, switch, etc.);
Various configurations, shapes, sizes and locations of IV label tab;
Packaging configuration that allows for sterile packaging to ensure device is sterile upon opening; and
Packaging configuration that allows for the potential to turn device on while still contained in the packaging (i.e., allows for device to be turned on and operated in the sterile environment through packaging) (FIG. 7, 11)

The invention includes a device for locating blood vessels of a patient, and is depicted in FIGS. 4-16. The device comprises at least one illumination strip including a top surface and a bottom surface and a securing device connected to the at least one illumination strip and configured to affix the bottom surface to the patient's skin. There is a light source disposed along the at least one illumination strip and directed toward an area on the patient's skin adjacent to the at least one illumination strip to illuminate the area on the patient's skin and enhance visibility of the patient's blood vessels.

There may be a plurality of interconnected illumination strips forming such as frame 16 in FIGS. 4 and 5. The frame defines a central aperture 28 about the area on the patient's skin and wherein the plurality of interconnected illumination strips each include a light source directed toward the central aperture. The securing member may comprise an adhesive disposed on the bottom surface of at least one illumination strip to affix the device to the patient's skin. The securing device may also comprise an adhesive disposed on the bottom surfaces of the illumination strips to affix the device to the patient's skin. In another embodiment, as shown in FIG. 6, there may be further included at least one additional illumination strip, such as strips 30a-d, which extend across the central aperture 28a of device 10a. The at least one illumination strip which extends across the central aperture may be curved in shape, as shown in FIG. 6.

The device may include an illumination device 32, FIG. 7, enclosed within sterile packaging 34 and able to be turned on to illuminate an area 36 on the patient's skin. In this view device 32 is vertically disposed (i.e. on an edge) to illuminate a narrow band or strip on the patient's arm.

As shown in FIG. 8, medical illumination device 40 may have LED lights 42 disposed on a strap 44 configured to be disposed about a limb, such as wrist 46, of the patient and secured by a fastener (not shown) to affix the device to the patient's skin. As can be seen blood vessels of a patient are shown to be illuminated in area 47 by the LED lights. As shown in FIG. 10, rectangular illumination device 60 may be secured to a patient's wrist with a strap 62.

In FIG. 9, illumination device 50 including rectangular illumination frame 52 is shown without a securement member. This embodiment is suited for use in one-time blood draws or injections where no IV needs to be secured in place.

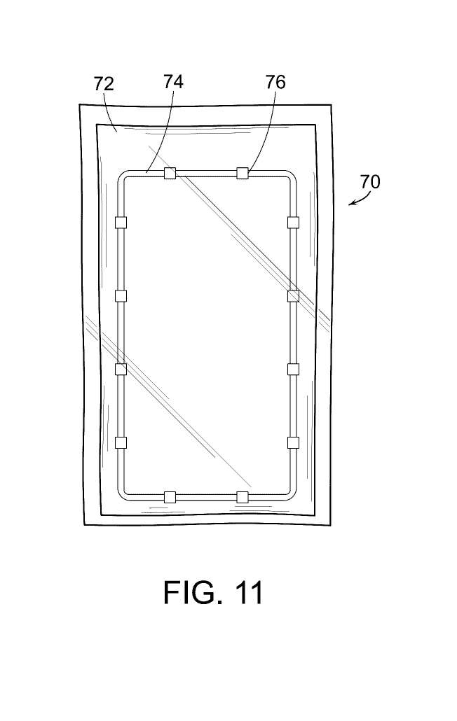

In FIG. 11, illumination device 70 is contained in sterile packaging 72 to ensure device is sterile upon opening. In this embodiment, illumination frame 74 and LEDs 76 may be activated within the packaging so the patient's veins may be illuminated in advance of removing the illumination device 70 from packaging 72. This concept is shown in use in FIG. 7.

Illumination frame 90, FIG. 12, includes elevated members 92 on which are disposed LEDs above the top surface of the illumination frame 90 such that they are able to project down toward aperture 94 and onto a patient's skin.

In one embodiment, as shown in FIG. 13, the bottom surface of illumination frame 100 is shown with adhesive affixed to only certain portions 102a-d of the frame 100 as opposed to being affixed in a continuous manner around the bottom surface of the frame.

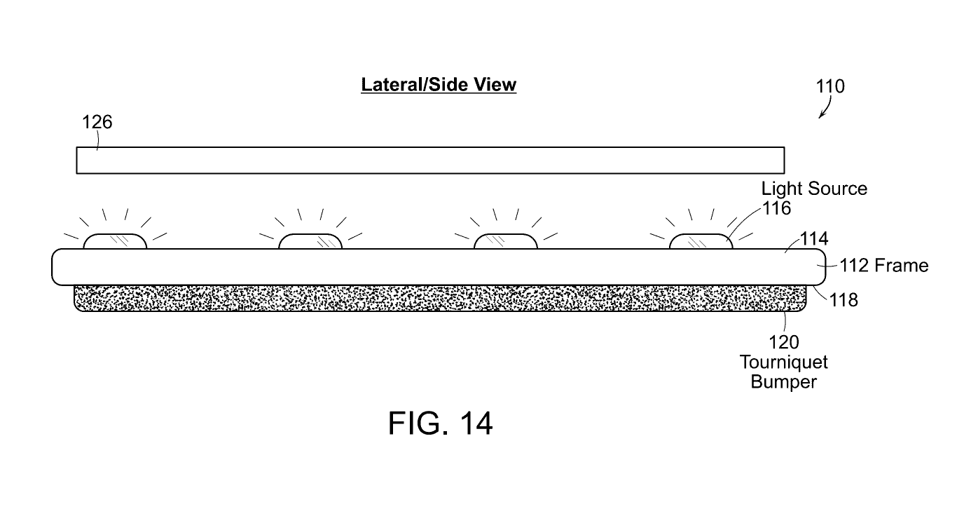

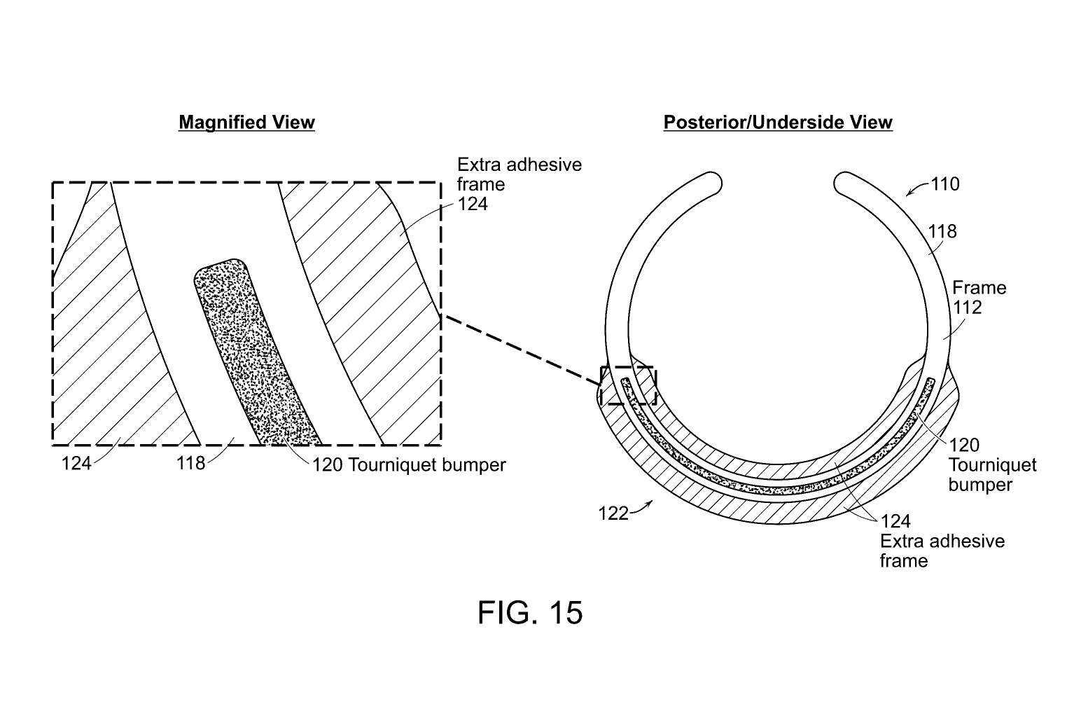

In another embodiment, as shown in FIGS. 14 and 15, there is an illumination device 110 including illumination frame 112 having a top surface 114 on which are disposed LEDs 116. On bottom surface 118 of illumination frame 112 is a bumper 120 which acts as a tourniquet to help pool blood in vessels, effectively dilating the vessels to render them more visible and suitable for venipuncture. To ensure proper function, bumper 120 should be formed of a material with sufficient stiffness such as plastic or other suitable material to press deeper into the tissue, thus, acting as a tourniquet to close vessels in order to pool blood and dilate the vessels. In FIG. 15, it can be seen that the illumination frame 112 is circular in shape; however, other shapes may be used as well. In one semi-circular region 122, of illumination frame 112, there is shown the bumper 120 on bottom surface 118. Adhesive 124 is provided on either side of bumper 120 to ensure the bumper is affixed to the patient's skin to provide a sufficient tourniquet effect. It should be noted that bumper 120 and adhesive could be extended a greater distance around the circular shape of frame 112 and need not be contained to semi-circular region 122.

In any of the above described embodiments, there may be included features to enhance illumination and/or visibility of the veins. This may include using a fluorescent filter to enhance illumination of the LEDs which may be included as part of the securement member 18 of FIG. 4 or in another suitable way. An example of this type of filter is shown in FIG. 14 as filter 126. Different wavelength LEDs, may be used, one wavelength to penetrate the skin and the other wavelength to allow the vein to absorb the light. Additionally minimization or canceling of the ambient light in the room or outdoors would be beneficial. This may be accomplished by filtering using a flap, a door, or a shade, with materials to filter ambient light. Filter 126 of FIG. 14 may be used for this purpose. Another means of filtering the ambient light is to have the user wear glasses (eye-wear) or use a filter (such as filter 126) that are selectively tinted to enhance the wavelengths that are absorbed by the blood vessels, thus, rendering them easier to view.

While preferred embodiments of the present invention have been shown and described herein, various modifications may be made thereto without departing from the inventive idea of the present invention. Accordingly, it is to be understood the present invention has been described by way of illustration and not limitation. Other embodiments are within the scope of the following claims.

What is claimed is:

1. A device for locating blood vessels of a patient, the device comprising:
At least one illumination strip including a top surface and a bottom surface;
A securing device connected to the at least one illumination strip and configured to affix the bottom surface to the patient's skin;
A light source disposed along the at least one illumination strip and directed toward an area on the patient's skin adjacent to the at least one illumination strip to illuminate the area on the patient's skin and enhance visibility of the patient's blood vessels.

2. The device of claim 1 further including a plurality of interconnected illumination strips.

3. The device of claim 2 wherein the plurality of interconnected illumination strips form a frame which defines a central aperture about the area on the patient's skin and wherein the plurality of interconnected illumination strips each include a light source directed toward the central aperture

4. The device of claim 1, wherein the securing device comprises an adhesive disposed on the bottom surface of at least one illumination strip to affix the device to the patient's skin.

5. The device of claim 3, wherein the securing device comprises an adhesive disposed on the bottom surfaces of the illumination strips to affix the device to the patient's skin.

6. The device of claim 3, wherein the securing device comprises a strap configured to be disposed about a limb of the patient and secured by a fastener to affix the device to the patient's skin.

7. The device of claim 5, further including a membrane hinged to a first portion of the frame and configured to be folded between an open position and a closed position to cover the central aperture and secure an intravenous line in place after insertion of the intravenous line line into the patient's blood vessel through the central aperture.

8. The device of claim 1, comprising a power source configured to provide power to said light source.

9. The device of claim 8, comprising a switch interconnected between the power source and the light source to activate the light source and illuminate the area on the patient's skin.

10. The device of claim 9, wherein the power source comprises a battery activated by a pull tab.

11. The device of claim 10, wherein the battery is affixed to a portion of at least one illumination strip.

12. The device of claim 11, wherein the battery includes a top surface on which a label is disposed to record patient information.

13. The device of claim 1, wherein the light source comprises a plurality of light emitting diodes.

14. The device of claim 13, wherein the light emitting diodes comprise printed strips of near infrared and other wavelengths of light emitting diodes.

15. The device of claim 3, wherein light source comprises a plurality of light emitting diodes disposed about the border of the frame.

16. The device of claim 1, wherein the at least one illumination strip comprises a fluorescent filter to enhance illumination.

17. The device of claim 3, wherein the bottom surfaces of the plurality of illumination strips contains an aseptic agent.

18. The device of claim 17, wherein the aseptic agent includes one or more of chlorhexidine gluconate, silver and zinc particles, and iodine.

19. The device of claim 7, wherein the membrane contains an aseptic agent.

20. The device of claim 19, wherein the aseptic agent includes one or more of chlorhexidine gluconate, silver and zinc particles, and iodine.

21. The device of claim 3, wherein the frame is rectangular in shape.

22. The device of claim 1, wherein the at least one illumination strip is in the form of a band which wraps around a limb of the patient.

23. The device of claim 1, wherein the light source is disposed above the top surface of the illumination strip

24. The device of claim 3, further including at least one additional illumination strip which extends across the central aperture.

25. The device of claim 23, wherein the at least one illumination strip which extend across the central aperture is curved in shape.

26. The device of claim 1 further including a sterile package which encompasses the device.

27. The device of claim 1 further including a bumper disposed on the bottom surface of the at least one illumination strip to acts as a tourniquet to pool blood in the blood vessels.

28. The device of claim 1 further including a fluorescent filter to enhance illumination of the light source.

29. The device of claim 1 further including an ambient light filter.

30. The device of claim 1 wherein the light source emits light a first wavelength suited to penetrate the skin and a second wavelength which allows the blood vessels to absorb the light.

31. The device of claim 30 further including a filter comprising materials to enhance viewing of the second wavelength.
